(19) United States
(12) Patent Application Publication
(10) Pub. No.: US 2021/0068712 A1
(43) Pub. Date: Mar. 11, 2021

(54) DETECTING THE END OF CYCLING ACTIVITIES ON A WEARABLE DEVICE

(71) Applicant: Apple Inc., Cupertino, CA (US)

(72) Inventors: Olivier P. HUMBLET, Queens, NY (US); James P. OCHS, San Francisco, CA (US); Vinay R. MAJJIGI, Mountain View, CA (US); Jonathan M. BEARD, San Jose, CA (US); Erin PAENG, Portland, OR (US); Karthik JAYARAMAN RAGHURAM, Mountain View, CA (US); Hung A. PHAM, Oakland, CA (US)

(73) Assignee: Apple Inc., Cupertino, CA (US)

(21) Appl. No.: 17/015,912

(22) Filed: Sep. 9, 2020

Related U.S. Application Data
(60) Provisional application No. 62/897,727, filed on Sep. 9, 2019.

(51) Int. Cl. A61B 5/11; A61B 5/0205; A61B 5/00

(52) U.S. Cl. CPC A61B 5/1123; A61B 5/0205; A61B 5/4866; A61B 5/02416; A61B 5/7285; A61B 5/681; A61B 5/1112

(57) ABSTRACT

Disclosed embodiments include wearable devices and techniques for detecting cycling activities and monitoring performance during cycling. By accurately and promptly detecting the end of cycling workouts automatically, the disclosure enables wearable devices to accurately calculate user performance information when users forget to stop recording a cycling activity session. In various embodiments, cycling activity detection techniques involve a cycling speed measure that incorporates terrain gradient determined based on pressure data. In various embodiments, the cycling activity detection techniques may distinguish between a temporary stop and an intentional stop using an estimated energy expenditure.

### CROSS REFERENCE TO RELATED APPLICATIONS

This application claims the benefit of U.S. Provisional Application Ser. No. 62/897,727 filed Sep. 9, 2019, the entire contents of which is hereby incorporated by reference.

### FIELD

The present disclosure relates generally to detecting the termination of cycling activities using a wearable device.

### BACKGROUND

A wearable device may be worn on the hand, wrist, or arm of a person when cycling. It may be desirable to track cycling activity by a user to promote exercise and for other health related reasons. Detecting the end point of a cycling activity is an essential component of accurately tracking cycling activities.

### SUMMARY

In one aspect disclosed herein are computerized methods for improving performance of a wearable device while recording a cycling activity, the methods including starting a cycling activity. Embodiments may also include receiving motion data of a user from a motion sensing module of the wearable device. Embodiments may also include measuring, by a heart rate sensing module of the wearable device, a heart rate of the user, the heart rate sensing module including a photoplethysmogram (PPG) sensor configured to be worn adjacent to the user's skin. Embodiments may also include calculating, by the one or more processor circuits, the user's performance information during the cycling activity, the performance information including a mechanical work rate and an energy expenditure rate. Embodiments may also include detecting, by the one or more processor circuits, an end of the cycling activity, the detecting the end of the cycling activity including comparing the mechanical work rate to a mechanical work rate threshold. Embodiments may also include in response to detecting a value for the mechanical work rate above the mechanical work rate threshold, calculating a difference between the mechanical work rate and the energy expenditure rate. Embodiments may also include determine the end of the cycling activity based on the difference between the mechanical work rate the energy expenditure rate.

Embodiments may also include receiving location data from a GPS module of the wearable device, the location data including a user speed of travel during the cycling activity. Embodiments may also include receiving atmospheric pressure data from a pressure sensor of the wearable device. Embodiments may also include calculating the mechanical work rate based the location data, the atmospheric pressure data, and the motion data.

In some embodiments, the mechanical work rate threshold is consistent with light exercise. Embodiments may also include detecting, by the one or more processor circuits, a stop during the cycling activity, the detecting the stop including receiving a value for the user speed of travel below an expected cycling speed. Embodiments may also include receiving motion data characterized as non-cycling motion.

Embodiments may also include estimating an energy expenditure rate at two or more points during the cycling activity based on the user heart rate. Embodiments may also include calculating a difference between a first energy expenditure rate estimated before the stop was detected and a second energy expenditure rate estimated after the stop was detected. Embodiments may also include detecting an intentional stop based on the difference between the first energy expenditure rate and the second energy expenditure rate.

Embodiments may also include upon detecting the intentional stop, ending the cycling activity and terminating calculation of the user's performance information. Embodiments may also include upon detecting the intentional stop, sending a confirmation request to a user to confirm the end of a cycling activity. Embodiments may also include detecting a temporary stop based on the difference between the first energy expenditure rate and the second energy expenditure rate. Embodiments may also include maintaining the cycling activity in response to detecting the temporary stop.

Embodiments may also include receiving a value for the user speed of travel within an acceptable range for an expected cycling speed and motion data characterized as a cycling motion. Embodiments may also include identifying a driving activity based on the difference between the mechanical work rate the energy expenditure rate. In some embodiments, the difference between the mechanical work rate the energy expenditure rate is equivalent to the mechanical work rate minus an energy expenditure rate consistent with light exercise.

Embodiments may also include in response to detecting the driving activity, ending the cycling activity, and stopping calculation of performance information. In some embodiments In some embodiments, the user speed of travel is calculated, by the one or more processor circuits, from GPS positioning data received from the GPS module. Embodiments may also include receiving the atmospheric pressure data from the pressure sensor of the wearable device. Embodiments may also include determining, by the one or more processor circuits, a grade describing a measure of steepness of terrain cycled on during the cycling activity. Embodiments may also include calculating, by the one or more processor circuits, elevation gained during the cycling activity using the grade.

Embodiments may also include detecting, during the cycling activity, a stepping motion within the motion data. In some embodiments, the stepping motion is distinct from a pedaling motion. Embodiments may also include extracting user steps included in the stepping motion using a user step model. Embodiments may also include based on user steps and the grade, determining mechanical work performed during the stepping motion. Embodiments may also include incorporating mechanical work performed by the user during the stepping motion into the mechanical work rate. In some embodiments, the performance information may include at least one of an overall distance traveled, a total cycling time, a speed, the elevation gained, a power output, and a number of calories burned.

In one aspect, disclosed herein are computerized methods for improving performance of a wearable device while recording a cycling activity, the methods including starting a cycling activity. Embodiments may also include receiving motion data of a user from a motion sensing module of the wearable device. Embodiments may also include measuring, by a heart rate sensing module of the wearable device, a heart rate of the user, the heart rate sensing module including a photoplethysmogram (PPG) sensor configured to be worn adjacent to the user's skin. Embodiments may also include calculating, by the one or more processor circuits, the user's performance information during the cycling activity, the performance information including a mechanical work rate and an energy expenditure rate. Embodiments may also include detecting, by the one or more processor circuits, an end of the cycling activity, the detecting the end of the cycling activity including comparing the mechanical work rate to a mechanical work rate threshold. Embodiments may also include in response to detecting a value for the mechanical work rate below the mechanical work rate threshold, ending the cycling workout and stopping calculation of the user's performance information. Embodiments may also include generating, by the one or more processor circuits, a notification including a request to end the cycling workout, the notification displayed on a display of the wearable device. In some embodiments, the motion data includes a step speed estimating a frequency of the user's lower body movements while performing pedal strokes during the cycling activity.

In one aspect, disclosed herein are systems for improving performance of a wearable device while recording a cycling activity, the systems including a motion sensing module configured to collect a user's motion data. Embodiments may also include a heart rate sensing module configured to measure a heart rate of the user. In some embodiments, the heart rate sensing module may include a photoplethysmogram (PPG) sensor and the PPG sensor is configured to be worn adjacent to the user's skin. Embodiments may also include one or more processor circuits in communication with the motion sensing module and the heart rate sensing module. In some embodiments, the one or more processor circuits are configured to execute instructions causing the processor circuits to start a cycling activity. In some embodiments the processor circuits may also calculate the user's performance information during the cycling activity, the performance information including a mechanical work rate and an energy expenditure rate. In some embodiments the processor circuits may also compare the mechanical work rate to a mechanical work rate threshold. In some embodiments, the processor circuits may also, in response to detecting a value for the mechanical work rate above the mechanical work rate threshold, calculate a difference between the mechanical work rate and the energy expenditure rate. In some embodiments, the processor circuits may also detect an end of the cycling activity based on the difference between the mechanical work rate and the energy expenditure rate.

Embodiments may also include a GPS module configured to measure location data including a user speed of travel during the cycling activity. Embodiments may also include a pressure sensor configured to measure atmospheric pressure data. In some embodiments, the processor circuits are further configured to, receive the location data and the pressure data. In some embodiments, the processor circuits may also calculate the mechanical work rate based the location data, the atmospheric pressure data, and the motion data.

### BRIEF DESCRIPTION OF THE DRAWINGS

Various objectives, features, and advantages of the disclosed subject matter can be more fully appreciated with reference to the following detailed description of the disclosed subject matter when considered in connection with the following drawings, in which like reference numerals identify like elements.

FIG. 1 is a diagram of an exemplary wearable device, according to embodiments of the disclosure.

FIG. 2 is a block diagram showing exemplary components that may be found within a wearable device, according to embodiments of the disclosure.

FIG. 3 is a flow chart illustrating a method for detecting the end of a cycling activity, according to embodiments of the disclosure.

FIGS. 4 and 5 are flow charts illustrating a method for using variable terrain gradient and speed measurements into more accurately track cycling activities, according to embodiments of the disclosure.

FIGS. 6-8 are flow charts illustrating methods for detecting an end of a cycling activity, according to embodiments of the disclosure.

FIG. 9 is a graph showing exemplary speed and heart rate date used to calculate energy discordance, according to embodiments of the disclosure.

FIG. 10 is a flow chart illustrating an exemplary method of providing a notification to a user confirming the end of a cycling activity, according to embodiments of the disclosure.

FIG. 11 is a flow chart illustrating an exemplary method of generating turn-by-turn navigation instructions during a cycling activity, according to embodiments of the disclosure.

### DESCRIPTION

The present disclosure describes systems and methods of detecting the end cycling activities using a wearable device. Cycling activities can occur indoors and outdoors, at a wide range of intensity levels and over varying terrain. For example, cycling activities can include racing rides having long period of continuous intense activity, commuter rides having shorter periods of less intense activity separated by stops for red lights and other traffic, stationary bike rides having a short to long period of continuous intense activity with no location change, and the like. Generalizable methods of detecting precise start and end points across the diverse range of cycling activities may be essential for accurately tracking the user's performance while cycling. Cycling activities may also occur over many hours. Due to, for example, size and cost constraints, the battery capacity of a wearable device, may make it impossible to run the device at full power continuously during a cycling activity without having to recharge the device's battery. Certain components of the device's battery, such as the main processor, Global Positioning System (GPS) receiver, and cellular module, all can draw a particularly high amount of power. Accordingly, one or more of these components may be powered down when they are not needed to track the cycling activity.

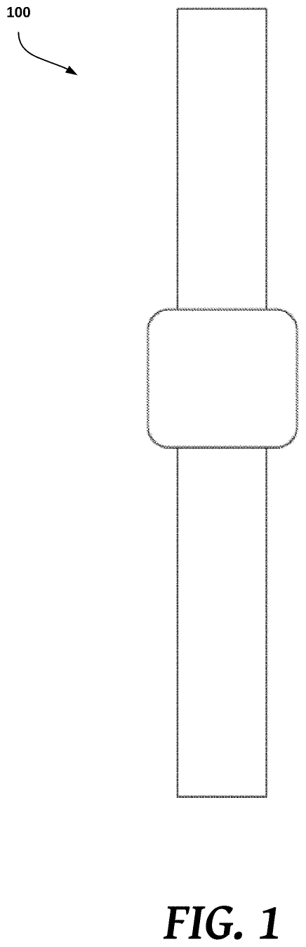

FIG. 1 shows an example of a wearable device 100 that may be worn by a cyclist (or “user”), in accordance with an embodiment of the present disclosure. In some embodiments, the wearable device 100 may be configured to be worn around the user's wrist using a strap (e.g., a watch strap).

As described in more detail below, the wearable device 100 may be configured to detect the user's cycling activity, calculate performance information of the user while cycling, and provide additional cycling-related functionality to the user. In particular, the wearable device 100 may use motion data obtained from motion sensors, heart rate data obtained from a heart rate sensing module, pressure data obtained from a pressure sensor and/or location information obtained from a GPS module to detect when the user stops cycling, makes a temporary stop during a cycling trip, cycles up a steep grade, cycles down a steep descent, makes a pedal stroke, coasts, cycles on a stationary bike, and/or performs other cycling related activity. The wearable device may use a variety of motion data, location information, and pressure data to determine a cycling specific measure for mechanical work rate. Heart rate data may be used by the wearable device to determine a user specific energy expenditure rate.

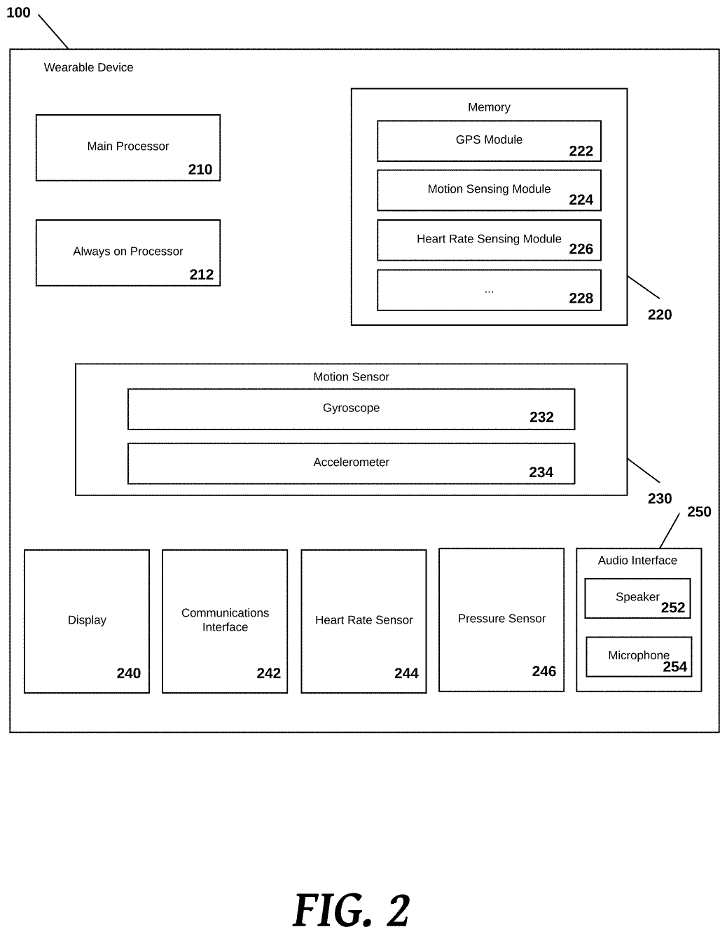

FIG. 2 depicts a block diagram of exemplary components that may be found within the wearable device 100 according to some embodiments of the present disclosure. In some embodiments, the wearable device 100 can include a main processor 210 (or “application processor” or “AP”), an always on processor 212 (or “AOP” or “motion co-processor”), a memory 220, one or more motion sensors 230, a display 240, an interface 242, a heart rate sensor 244, and a pressure sensor 246. The wearable device 100 may include additional modules, fewer modules, or any other suitable combination of modules that perform any suitable operation or combination of operations.

In some embodiments, main processor 210 can include one or more cores and can accommodate one or more threads to run various applications and modules. Software can run on main processor 210 capable of executing computer instructions or computer code. The main processor 210 can also be implemented in hardware using an application specific integrated circuit (ASIC), programmable logic array (PLA), field programmable gate array (FPGA), or any other integrated circuit.

In some embodiments, wearable device 100 can also include an always on processor 212 which may draw less power than the main processor 210. Whereas the main processor 210 may be configured for general purpose computations and communications, the always on processor 212 may be configured to perform a relatively limited set of tasks, such as receiving and processing data from motion sensor 230, heart rate sensor 244, pressure sensor 246, and other modules within the wearable device 100. In many embodiments, the main processor 210 may be powered down at certain times to conserve battery charge, while the always on processor 212 remains powered on. Always on processor 212 may control when the main processor 210 is powered on or off.

Memory 220 can be a non-transitory computer readable medium, flash memory, a magnetic disk drive, an optical drive, a programmable read-only memory (PROM), a read-only memory (ROM), or any other memory or combination of memories. Memory 220 can include one or more modules 222-228.

The main processor 210 and/or always on processor 212 can be configured to run one or more modules 222-228 stored in memory 220 that are configured to cause main processor 210 or always on processor 212 to perform various steps that are discussed throughout the present disclosure.

In some embodiments, the wearable device 100 can include one or more motion sensors 230. For example, motion sensors 230 can include a gyroscope 232 and an accelerometer 234. In some embodiments, accelerometer 234 may be a three-axis accelerometer that measures linear acceleration in up to three-dimensions (for example, x-axis, y-axis, and z-axis). In some embodiments, gyroscope 232 may be a three-axis gyroscope that measures rotational data, such as rotational movement and/or angular velocity, in up to three-dimensions (for example, yaw, pitch, and roll). In some embodiments, accelerometer 234 may be a microelectromechanical system (MEMS) accelerometer, and gyroscope 232 may be an MEMS gyroscope. Main processor 210 or always on processor 212 of wearable device 100 may receive motion information from one or more motion sensors 230 to track acceleration, rotation, position, or orientation information of wearable device 100 in six degrees of freedom through three-dimensional space.

In some embodiments, the wearable device 100 may include other types of sensors in addition to accelerometer 234 and gyroscope 232. For example, the wearable device 100 may include a pressure sensor 246 (e.g., an altimeter or barometer) and/or a location sensor (e.g., a Global Positioning System (GPS) sensor).

The wearable device 100 may also include a display 240. The display 240 may be a screen, such as a crystalline (e.g., sapphire) or glass touchscreen, configured to provide output to the user as well as receive input from the user via touch. For example, the display 240 may be configured to display a current heart rate or daily average energy expenditure. The display 240 may receive input from the user to select, for example, which information should be displayed, or ending a physical activity (e.g., ending a session), such as a swimming session, a running session, or a cycling session. In some embodiments, wearable device 100 may present output to the user in other ways, such as by producing sound with a speaker 252, and wearable device 100 may receive input from the user in other ways, such as by receiving voice commands via a microphone 254.

In various embodiments, wearable device 100 may communicate with external devices via an interface 242, including a configuration to present output to a user or receive input from a user. The interface 242 may be a wireless interface. The wireless interface may be a standard Bluetooth® (IEEE 802.15) interface, such as Bluetooth® v4.0, also known as “Bluetooth low energy.” In various embodiments, the interface may operate according to a cellphone network protocol such as Long Term Evolution (LTE™) or a Wi-Fi (IEEE 802.11) protocol. In various embodiments, the interface 242 may include wired interfaces, such as a headphone jack or bus connector (e.g., Lightning®, Thunderbolt™, USB, etc.).

Wearable device 100 can measure an individual's current heart rate from a heart rate sensor 244. The heart rate sensor 244 may also be configured to determine a confidence level indicating a relative likelihood of an accuracy of a given heart rate measurement. In various embodiments, a traditional heart rate monitor may be used and may communicate with wearable device 100 through a near field communication method (e.g., Bluetooth).

In various embodiments, the wearable device 100 can include a photoplethysmogram (PPG) sensor. PPG is a technique for measuring a person's heart rate by optically measuring changes in the person's blood flow at a specific location. PPG can be implemented in many different types of devices in various forms and shapes. For example, a PPG sensor can be implemented in a wearable device 100 in the form of a wrist strap, which a user can wear around the wrist. The PPG sensor can optically measure the blood flow at the wrist. Based on the blood flow information, the wrist strap or another connected device can derive the person's heart rate.

The wearable device 100 may be configured to communicate with a companion device, such as a smartphone. In various embodiments, wearable device 100 may be configured to communicate with other external devices, such as a notebook or desktop computer, tablet, headphones, Bluetooth headset, etc.

The modules described above are examples, and embodiments of wearable device 100 may include other modules not shown. For example, some embodiments of wearable device 100 may include a rechargeable battery (e.g., a lithium-ion battery), a microphone array, one or more cameras, two or more speakers, a watchband, water-resistant casing or coating, etc. In some embodiments, all modules within wearable device 100 can be electrically and/or mechanically coupled together. In some embodiments, main processor 210 and or always on processor 212 can coordinate the communication among each module.

In various embodiments, the wearable device 100 may use sensed and collected motion information to predict a user's activity. Examples of activities may include, but are not limited to, walking, running, cycling, swimming, skiing, etc. Wearable device 100 may also be able to predict or otherwise detect when a user is sedentary (e.g., sleeping, sitting, standing still, driving or otherwise controlling a vehicle, etc.). Wearable device 100 may use a variety of motion information to predict a user's activity.

Wearable device 100 may use a variety of heuristics, algorithms, or other techniques to predict the user's activity. Wearable device 100 may also estimate a confidence level (e.g., percentage likelihood, degree of accuracy, etc.) associated with a particular prediction (e.g., 90% likelihood that the user is cycling) or predictions (e.g., 60% likelihood that the user is cycling and 40% likelihood that the user is performing some other activity).

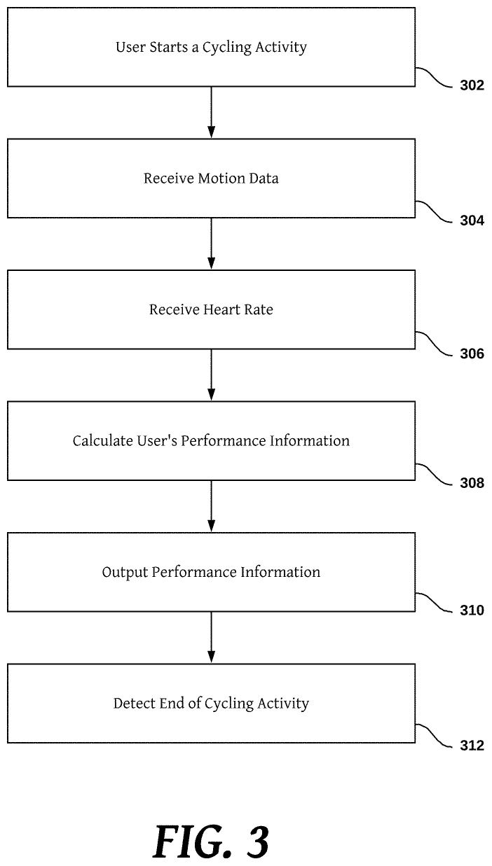

FIG. 3 shows an exemplary method for tracking the performance of a user during a cycling activity. At step 302, a user starts a cycling activity. At step 304, motion data of the user is received, for example, from a motion sensor of the wearable device shown in FIG. 2. The motion data may include accelerometer, gyroscope, GPS, speed, or pressure data according to some embodiments. At step 306, heart rate data of the user is received, for example, from heart rate sensor of the wearable device shown in FIG. 2. At step 308, motion data and/or heart rate data received by the wearable device may be recorded and used to calculate user performance information.

In various embodiments, performance information may include pedaling cadence, power output, average power output, mechanical work rate, energy expenditure rate, heart rate, average heart rate, gradient of terrain cycled, average gradient of terrain cycled during an uphill or downhill segment, distance traveled, total cycling time, cycling speed, average speed, elevation gained, ascent rate, calories burned, and the like. At step 310, performance information calculated by the wearable device may be output to a user, for example, rendered on a wearable device display (e.g., a crystalline or glass touchscreen). In various embodiments, the wearable device may provide additional functionality after a cycling activity begins, for example, providing turn by turn navigation instructions to a desired destination, detecting a temporary stop during a cycling activity, detecting a stationary cycling activity, syncing a cycling activity with another activity tracking application, detecting the presence of one or more cycling companions accompanying a user during a cycling activity (e.g., other cyclists riding in a group with the user), and the like.

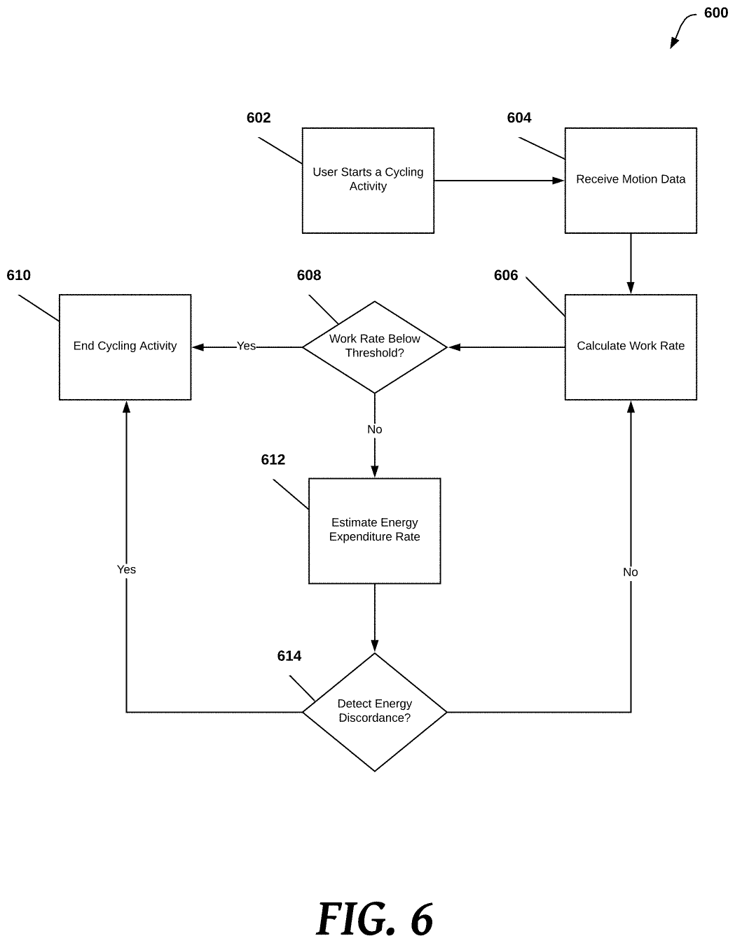

At step 312, a wearable device may detect the end of a cycling activity by based on motion data and/or heart rate data. In various embodiments, the wearable device may compare one or more measurements included in performance information to confirm the end of a cycling activity. FIGS. 6-8 below describe how a wearable device determines an ending point for a cycling activity in more detail. Once a wearable device determines a cycling activity has ended, it may stop calculating performance information and/or may aggregate performance information calculated during the cycling activity into cycling session dataset that may be output to the user, for example, rendered on a wearable device display, uploaded to an activity tracking application, and the like.

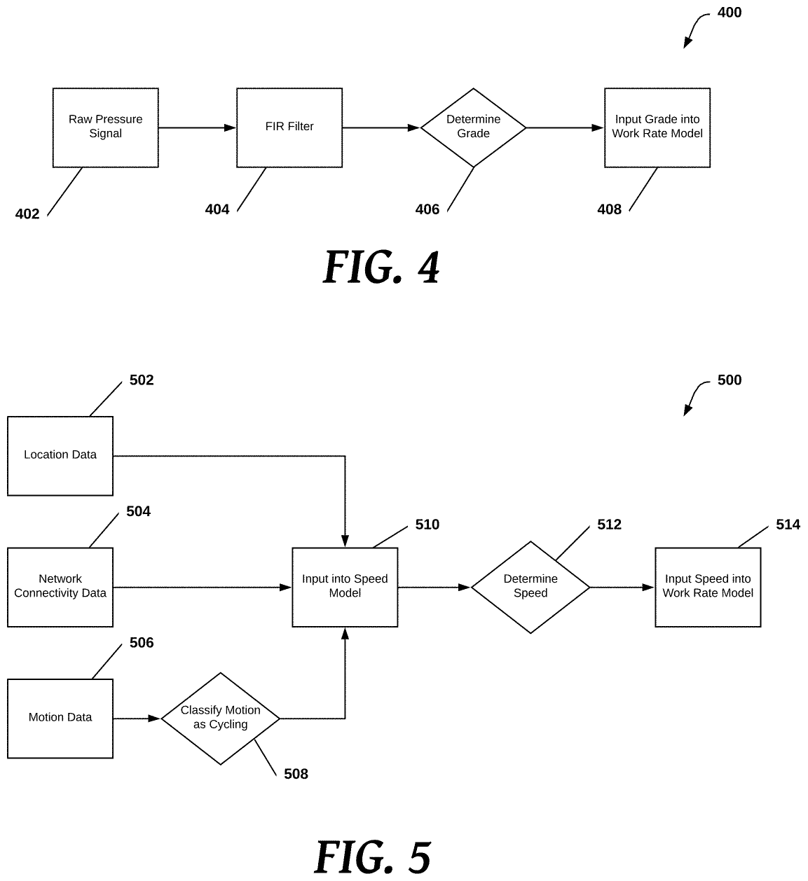

FIGS. 4-5 illustrate exemplary methods that can be used to calculate a mechanical work rate of a user during a cycling activity. The mechanical work rate may be calculated using a cycling specific work rate model that incorporates one or more aspects particular to cycling in mechanical work rate calculations. In various embodiments, the one or more aspects particular to cycling that can be incorporated into mechanical work rate calculations include cycling speed, step speed, and terrain gradient. FIG. 4 describes how gradient information may be incorporated into the work rate model and FIG. 5 describes how cycling speed may be incorporated into the work rate model.

FIG. 4 illustrates an exemplary method for calculating terrain gradient information using pressure data obtained from a pressure sensor of the wearable device. At step 402, the wearable device receives raw pressure data, for example, a raw pressure signal from the pressure sensor. The raw pressure signal can be filtered to improve data quality, at step 404. Pressure data, in particular, can be noisy making it difficult to extract reliable, accurate information from the raw signal. In various embodiments, a filter (e.g., a finite impulse response (FIR) filter) may be used to smooth the raw pressure signal in order to get a more accurate estimate of elevation change. At step 406, one or more filtered pressure signals taken at a first time point may be compared to one or more filtered pressure signals for a subsequent time point to determine if a user is cycling on flat or sloped ground. For example, an increase in pressure from the first time point to the second time point would indicate travel on a negative slope (e.g., a descending down a hill); a decrease in pressure from the first time point to the second time point would indicate travel on a positive slop (e.g., ascending up a hill); and no change in pressure form the first time point to the second time point would indicate travel on a flat ground. Using the comparison between filtered pressure signals, the wearable device may calculate a gradient (e.g., gradient output in percent grade) describing the angle of ascent or decent the user is cycling on at any given point in time during the cycling activity. The change in pressure may be continuously calculated during the cycling activity to constantly determine the gradient the user is cycling on at all times during the cycling activity. At step 408, the gradient measurements may be incorporated into a cycling specific work rate model for calculating mechanical work rate to accurately model an increased mechanical work rate when cycling uphill on terrain having a positive gradient relative to cycling on flat ground or cycling downhill; and a decreased mechanical work rate when cycling downhill on terrain having a negative gradient relative to cycling on flat ground or cycling uphill. The mechanical work rate model for cycling uphill and or cycling downhill can be specific to a user and can be derived by surveying a plurality of datasets of previous cycling activities completed by the user and or surveying a plurality of datasets of completed cycling activities performed by users having one or more attributes in common with the user.

FIG. 5 illustrates an exemplary method for calculating cycling speed data of a user during a cycling activity. A wearable device may calculate cycling speed using one or more data streams received from one or more sensors and/or modules included in the wearable device. Cycling speed may then be incorporated into a work rate model for determining the mechanical work rate of a user during a cycling activity.

At step 502, the wearable device receives location data from a GPS module. Location data may processed by a speed model that determines cycling speed, at step 510. In various embodiments, a speed model may determine cycling speed from location data (e.g., GPS data) by calculating a travel time between two or more locations having a known distance and determining a speed measurement (e.g., velocity, rate of travel, and the like) using a ratio of the known distance to the travel time. At step 506, the wearable devices receives motion data from a motion sensor. At step 508, motion data may be classified as a cycling motion by the wearable device. In various embodiments, the wearable device may classify motion data as cycling motion based on a cycling motion profile generated from surveying a plurality of motion datasets measured during known cycling activities, known non-cycling activities, and unclassified activities. Once motion data is classified as cycling motion, at step 510, motion data may be input into the speed model to supplement location data to improve the accuracy and/or reliability of the speed measurements output by the speed model.

During the cycling activity, the wearable device may also detect a stepping motion within the motion data. For example, when a user is cycling over a steep grade or other hazardous terrain and decides to stop cycling and push her bike. The stepping motion may be distinct from a pedaling motion and may be classified as a walking and or running activity by the wearable device. In response to detecting the stepping motion, the wearable device may extract user steps included in the stepping motion using a step model. The step model may be particular to the user's stride length and or other characteristics of the user. The step model may be determined by surveying a plurality of datasets including step motion data from walking and or running activities having known step counts. The walking and or running activities included in the plurality of datasets may be performed by the user and or a group of users having one or more characteristics in common with the user. Based on user steps and the grade, the wearable device may determine the mechanical work performed during the stepping motion. The mechanical work rate for the stepping motion may then be incorporated into the user's mechanical work rate during the cycling activity.

In some situations, generating speed measurements from location data may restrict the battery life of the wearable device by consuming significant power and/or compute resources (e.g., processing capacity, memory, network communications, and the like). To prolong the battery life of the wearable device, speed measurements may be generated by the one or more speed models using network connectivity data and/or motion data. At step 504, the wearable device may receive network connectivity data from a communications interface. In various embodiments, network connectivity data may include signal strength, connection history, location of network access point, timestamps of when a connection request was received, timestamps of when a connection was established, connection type, and the like. At step 510, connection data may be input into one or more speed models to generate cycling speed measurements. In various embodiments, the one or more speed models may determine cycling speed measurements by comparing the signal strength received by the wearable device from two or more network access points having a known distance to calculate a first location of the user at a first point in time. A second location for a user at a second point in time may then be determined by comparing the signal strengths received by the wearable device at the second point in time. Cycling speed measurements may then be calculated from the ratio of the distance between the first location and the second location to the time difference between the first point in time and the second point in time. In various embodiments, cycling speed measurements may be calculated from network connectivity data based on the network refresh rate between two network access points having a known location. The network refresh rate may be calculated using the time required by the wearable device to change from connecting to a first network access point to connecting to a second network access point. Using the network refresh rate, signal strengths of each network connection, and the distance between the first network access point having a known location and the second access point having a different known location, the one or more speed models may determine the cycling speed of a user.

In various embodiments, the one or more speed models may incorporate motion data to enhance the accuracy and/or reliability of the cycling speed measurements calculated from network connectivity data. In various embodiments, the one or more speed models may use any combination of motion data, network connectivity, and/or location data to determine cycling speed of a user.

FIGS. 6-8 illustrate exemplary methods of detecting the end of cycling activities. FIG. 6 illustrates an exemplary method of terminating a cycling activity. At step 602, the user starts a cycling activity. At step 604, the wearable device may receive motion data from one or more motion sensors. In various embodiments, the wearable device may also measure heart rate data of the user using one or more heart rate sensors. The heart rate sensor can be a PPG sensor and the wearable device may receive and/or process heart rate data from the PPG sensor according to techniques described in U.S. patent application Ser. No. 15/692,736, filed on Aug. 31, 2017, and entitled “SYSTEMS AND METHODS FOR DETERMINING AN INTENISITY LEVEL OF AN EXERCISE USING PHOTOPLETHYSMOGRAM (PPG),” which patent application is incorporated herein in its entirety. The wearable device may compare the heart rate data received from the heart rate sensor to a resting heart rate threshold (e.g., 30-70 beats per minute (bbm)) to determine the user is active. The resting heart rate threshold may depend on characteristics of the user including age, fitness level, level of fatigue, and the like. The resting heart rate threshold may be specific to a user and may be determined by surveying a plurality of datasets including heart rate data measured during cycling activities and or other activities (e.g., walking, running, swimming, skiing, yoga, and the like) completed by the user and or a group of users having one or more characteristics in common with the user.

Once a cycling activity is started, the wearable device may calculate performance information of a user including a mechanical work rate and/or an energy expenditure rate. Based on performance information calculated during the cycling activity, the wearable device may determine when the user stops cycling. Stops detected by the wearable device may be classified as temporary and/or permanent.

To detect a stop, the wearable device may calculate a mechanical work rate of a user at step 606. The mechanical work rate may be calculated using a cycling work rate model that may incorporate some combination of cycling speed, pedaling speed (e.g., step speed during cycling, cadence, and the like), and/or terrain gradient. In various embodiments, the cycling work rate model may also incorporate step speed and cadence into work rate computations to account for situations track work performed by a user while pushing her bike. At step 608, the wearable device may compare the mechanical work rate calculated for a user to a work rate threshold (e.g., 50-100 joules/second, 50-100 Watts, 2.1 to 2.9 metabolic equivalent (METs), and the like). The mechanical work rate threshold may be consistent with light exercise. In various embodiments, the work rate threshold may be specific to a user and may be determined by surveying, for example, a plurality of datasets including an average, minimum, and/or maximum work rate achieved during previous cycling activities. The previous cycling activities included in the plurality of datasets may be performed by the user and or a group of users having one or more characteristics in common with the user. In various embodiments, the work rate threshold may be a global work rate threshold generalizable across multiple users. The global work rate threshold may be determined, for example, by surveying a plurality of datasets including the mechanical work rate measured during cycling activities and or other activities (e.g., walking, running, swimming, skiing, yoga, and the like). The activities included in the plurality of datasets may be performed by the user and or a group of users having one or more characteristics in common with the user. To accurately detect all cycling activities including low intensity cycling activities, the work rate threshold may be consistent with a work rate achieved during light exercise (i.e., between 2.1 and 2.9 METs). If the mechanical work rate is below the mechanical work rate threshold, the wearable device may determine the user has stopped cycling and may detect an end of a cycling activity, at step 610. If the mechanical work rate is above the mechanical work rate threshold, the wearable device may determine the user is cycling and/or still active and may continue to calculate performance information including energy expenditure rate. At step 612, the wearable device may calculate the energy expenditure rate of a user using a cardio-respiratory model that may be specific to one or more characteristics (e.g., age, weight, cardiovascular health, fitness level, and the like) of the user.

The difference between mechanical work rate and the energy expenditure rate may be calculated to determine an energy discordance. In various embodiments, the energy discordance may be used to identify situations in which the work-output and the energy expenditure do not agree. Accordingly, the energy discordance (i.e., situations in which the work-output reflected by the mechanical work rate is consistently different from the energy expenditure) may be interpreted by the wearable device as an end point for a workout activity. The energy discordance may be detected at step 614, for example, when the work performed by the user (i.e., the mechanical work rate) is inconsistent with the amount of cardio-vascular exertion (i.e., the energy expenditure). To determine the energy discordance, the wearable device may compare the difference between the mechanical work rate and the energy expenditure rate to an energy discordance threshold (e.g., 1-10). The difference between the mechanical work rate and the energy expenditure rate may be calculated for a predetermined period of time and or continuously calculated during the cycling activity. If the difference between the mechanical work rate and the energy expenditure rate is above the energy discordance threshold for a particular period of time, the wearable device may detect an energy discordance. In response to detecting an energy discordance at step 614, the wearable device may detect an end point for a cycling activity and may stop calculating performance information and or end the cycling activity at step 610. If the difference between the mechanical work rate and the energy expenditure rate is below the energy discordance threshold, the wearable device may not detect an energy discordance and may maintain the cycling activity by continuing to calculate mechanical work rate and other performance information at step 606.

The wearable device may monitor the work-output, energy expenditure, and or energy discordance continuously during the cycling activity to improve the accuracy of detecting the end point for the cycling activity. The wearable device may also determine if an energy discordance can be observed in the performance information in response to a triggering event. For example, the triggering event may include energy expenditure, mechanical work rate, work-output, speed, and the like dropping and or rising to a particular level. The energy discordance threshold used to detect the energy discordance may be particular to a user and or a group of users having one or more characteristics in common with the user. For example, the energy discordance threshold may be determined by surveying a plurality of datasets including measurements of the difference between then mechanical work rate and the energy expenditure rate taken at various points (i.e., before, during, at a known stopping point, after, and the like) of cycling activities and other activity types. The activities included in the plurality of datasets may be performed by the user and or a group of users having one or more characteristics in common with the user.

In various embodiments, the energy discordance may be used to detect situations when a user has ended a cycling activity and begun driving. In these situations, the mechanical work rate far exceeds the user's energy expenditure because the mechanical work performed by the car exceeds the maximum mechanical work rate achievable from users performing cycling activities and the user's energy expenditure is relatively low because she is stationary in the car and not exercising. In this situation, the difference between mechanical work rate and energy expenditure rate exceeds the energy discordance threshold causing and energy discordance to be detected. To prevent, the mechanical work observed during driving from being incorporated into the cycling activity, the detection of the energy discordance is used to determine the end of a cycling activity.

In various embodiments, energy discordance may be used to distinguish situations when a user is cycling through a location where GPS data is not available (e.g., a tunnel, indoors during a stationary cycling activity, and the like) from when a user has stopped a cycling activity. When GPS data is not available, the calculated mechanical work rate is held over from the last available speed measurements. If the difference between mechanical work rate and energy expenditure rate exceeds the energy discordance threshold, an energy discordance is detected. In response to detecting the energy discordance, the wearable device determines the cycling activity has ended even though current GPS data is not available and previous GPS data indicates the user is still cycling. If the energy expenditure rate remains elevated so that the difference between the mechanical work rate and the energy expenditure rate is below the energy discordance threshold, the wearable devices determines the user is cycling and maintains the cycling activity session. The energy discordance threshold for situations when GPS data is unavailable may be equal to the user's average mechanical work rate less a light exercise energy expenditure (e.g., under 3 metabolic equivalents (METs)).

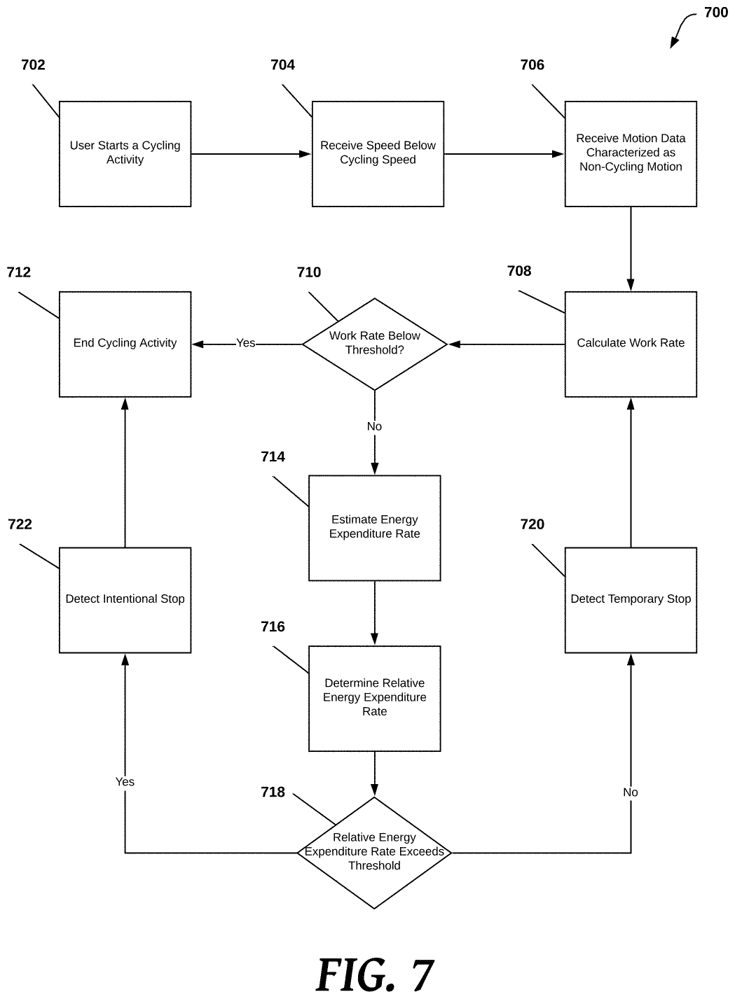

FIG. 7 illustrates an exemplary method for detecting a stop during a cycling activity. After detecting a stop, the method may distinguish between a temporary stop (e.g., a stop at a traffic light or stop sign, a stop for directions, and the like) and an intentional stop (e.g., the end of a cycling activity). At step 702, a user starts a cycling activity. At step 704, as the user begins to slow down for a stop, the wearable device receives a cycling speed from the one or more speed models that is below the expected cycling speed of the user. In various embodiments, the expected cycling speed of the user may be dependent on the terrain gradient information calculated from pressure data obtained from the pressure sensor. Specifically, the expected cycling speed uphill (e.g., a 6% grade) may be lower than the expected cycling speed on flat ground or downhill and the expected cycling speed downhill (e.g., a −6% grade) may be higher than the expected cycling speed on flat ground or uphill. The expected cycling speed for a user may be determined by surveying a plurality of datasets of previous cycling activities including speed measurements taken over a wide range of gradients (e.g., between −20% or greater downhills grades and 20% or greater uphill grades.) The previous cycling activities included in the plurality of datasets may be performed by the user and or a group of users having one or more characteristics in common with the user. At step 706, the wearable device may receive motion data characterized as non-cycling motion as the user stops pedaling and or takes her hands off of the handle bars during the stop.

In various embodiments, the cycling speed and motion data for a temporary stop may be identical to the cycling speed and motion data observed after a user finishes a cycling activity. At step 708, the wearable device may calculate the mechanical work rate of a user. In various embodiments, at step 710, the wearable device may compare the mechanical work rate to a mechanical work rate threshold to detect an end of a cycling activity without having to calculate energy expenditure. For example, if the mechanical work rate is below the mechanical work rate threshold, the wearable device may determine a cycling activity has ended, at step 712. The mechanical work rate threshold may be determined by surveying a plurality of datasets including mechanical work rates measured during cycling activities and or other activity types. The activities included in the plurality of datasets may be performed by the user and or a group of users having one or more characteristics in common with the user.

If the mechanical work rate is below the mechanical work rate threshold, there is a possibility the cycling activity is still ongoing and energy expenditure is analyzed. To distinguish a temporary stop from the end of a cycling activity, the wearable device may monitor the change in energy expenditure after the stop is detected. At step 714, the wearable device may estimate the energy expenditure rate of a user using heart rate data. The wearable device may continuously estimate the energy expenditure rate of a user during the cycling activity session to track the changes in the energy expenditure rate over time. In various embodiments, the energy expenditure rate of a user may be calculated based on user characteristics including age, gender, fitness level, and the like.

At step 716, the wearable device may compute the relative energy expenditure rate by calculating the difference between the energy expenditure rate at two different time points during the cycling activity. For example, a relative energy expenditure rate may be calculated by computing the difference between a first energy expenditure rate calculated at a point in time during the cycling activity before a stop was detected and a second energy expenditure rate calculated at a point in time after the stop was detected. At step 718, the relative energy expenditure rate may be compared to an energy expenditure rate threshold to distinguish between a temporary stop and an intentional stop. The relative energy expenditure rate threshold may be consistent with a gradual decay in energy expenditure rate from level consistent with high to light exercise (e.g., greater than 3.0 METs for medium to intense exercise and between 2.1-2.9 METs for light exercise) to a level consistent with a non-exercise, sedentary state (e.g., below 2.1 METs). In various embodiments, a series of two more comparisons between a current calculated relative energy expenditure rate and the relative energy expenditure rate threshold may be made after a stop is detected to track a gradual decay of in energy expenditure needed to detect an intentional stop. The relative energy expenditure rate threshold may be for example, −1.0 METs to −0.1 METs. The relative energy expenditure rate threshold may be determined by surveying a plurality of datasets including cycling activities and other activity types having known stopping points and a measure of decay in energy expenditure rate observed after the stopping points. The plurality of datasets may include activities performed by the user and or a group of users having one or more characteristics in common with the user. Detecting one or more relative energy expenditure rates below the relative energy expenditure threshold may cause the wearable device to detect an intentional stop at step 722 and end the cycling activity session at step 712. For example, an intentional stop may be detected by observing 3 relative energy expenditure rates within or below the relative energy expenditure rate threshold (e.g., −1.1 METs, −0.2 METs, and −0.4 METs) during the first 1 to 5 minutes after a stop was detected.

If at step 718, the relative energy expenditure rate exceeds the relative energy expenditure rate threshold, a temporary stop may be detected and the cycling activity may be maintained causing the wearable device to continue to calculate user performance information including work rate at step 708. In various embodiments, a series of two more comparisons between a current calculated relative energy expenditure rate and the relative energy expenditure rate threshold may be made after a stop is detected to detect a temporary stop. Detecting one or more relative energy expenditure rates above the relative energy expenditure threshold may cause the wearable device to detect a temporary stop at step 720 and maintain the cycling activity. For example, a temporary stop may be detected by observing 3 relative energy expenditure rates above the relative energy expenditure rate threshold (e.g., +1.1 METs, +0.2 METs, and +0.4 METs) during the first 1 to 5 minutes after a stop was detected.

In various embodiments, stationary cycling activities may be detected using the method described above. For example, situations having a speed measurements below an expected cycling speed and mechanical work rates above a work rate threshold and energy expenditure rates above a relative energy expenditure rate threshold may be recognized by the wearable device as stationary cycling activities.

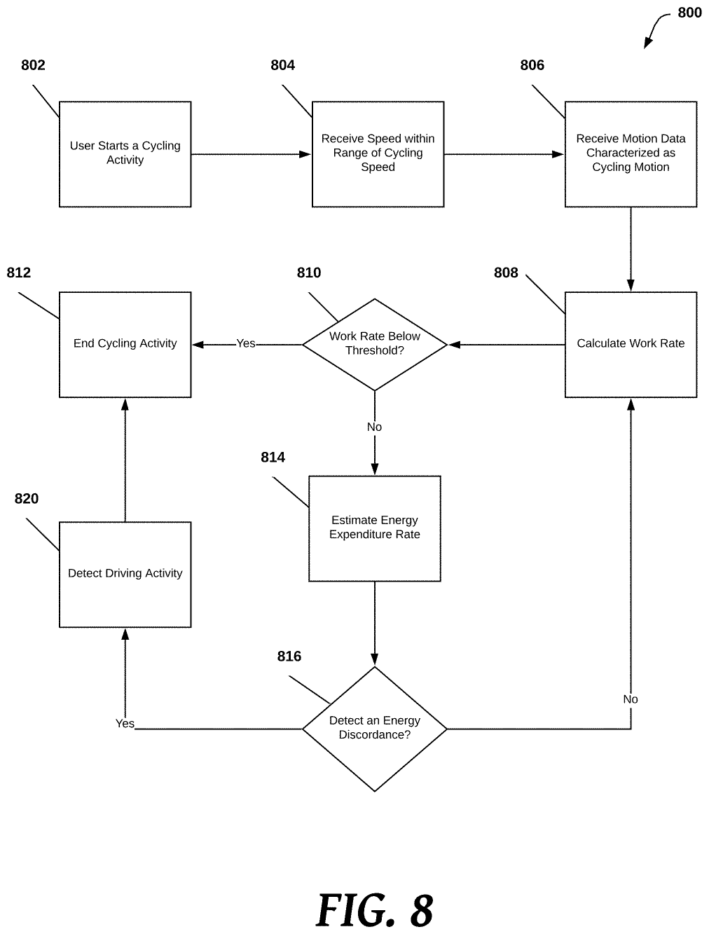

FIG. 8 illustrates an exemplary method for detecting a driving activity. In various embodiments, the speed of travel and motion data received during driving may be similar to the speed of travel and motion data received during cycling. Therefore, energy discordance may be used to distinguish driving activities from cycling activities.

At step 802, the wearable device detects a starting point for a cycling activity using heart rate data and/or motion data and begins a cycling activity. In situations where a user starts driving after performing an exercise activity and/or has an elevated heart rate when driving, the heart rate data and motion data during driving activities and cycling activities may be similar. At step 804, the wearable device may receive a cycling speed within the range of expected cycling speeds. Even though the user is driving, the driving speed may be similar to a cycling speed particularly when the user driving at slow speeds or a user has a high fitness level and/or cycles fast when traveling downhill. The hand position of the user detected by the wearable device may also be similar when driving relative to cycling. For example, the hand motion and position observed while a user holds the handle bars and steers the bike may be similar to the hand motion and position observed during driving when a user holds the steering wheel and steers the car. Therefore, at step 806, the wearable device may receive motion data characterized as cycling motion even when the user is driving because the wrist and/or arm pose of the user may be similar during cycling and driving. During the cycling activity, the wearable device may detect performance information including a mechanical work rate of the user at step 808. At step 810, the wearable device may compare the mechanical work rate to a work rate threshold. If the mechanical work rate is below the work rate threshold, the wearable device may determine the cycling activity has ended and may stop calculating performance information. If the mechanical work rate is above the work rate threshold, as it may be during driving due to the high speed of travel, the wearable device may determine if the user is driving based on energy discordance.

At step 814, the wearable device may estimate the energy expenditure rate of the user during the cycling activity. At step 816, the wearable device may detect an energy discordance based on the difference between mechanical work rate and the energy expenditure rate. To detect an energy discordance, the wearable device may calculate the difference between the mechanical work rate and the energy expenditure rate and compare the calculated difference to an energy discordance threshold. If the difference between the mechanical work rate and the energy expenditure rate is above the energy discordance threshold, the wearable device may detect an energy discordance. In response to detecting the energy discordance, the wearable device may detect a driving activity at step 820. In response to detecting the driving activity, the wearable device may end of the cycling activity and stop calculating performance information. The wearable device may also not resume an existing cycling activity and or may not begin a new cycling activity in response to detected the driving activity. If the difference between the mechanical work rate and the energy expenditure rate is below the energy discordance threshold, the wearable device may not detect an energy discordance. In response to not detecting an energy discordance, the wearable device may maintain the cycling activity and continue to calculate performance information until the speed drops below or increases above the range of expected cycling speeds and/or the wearable device receives motion data indicative of a non-cycling motion.

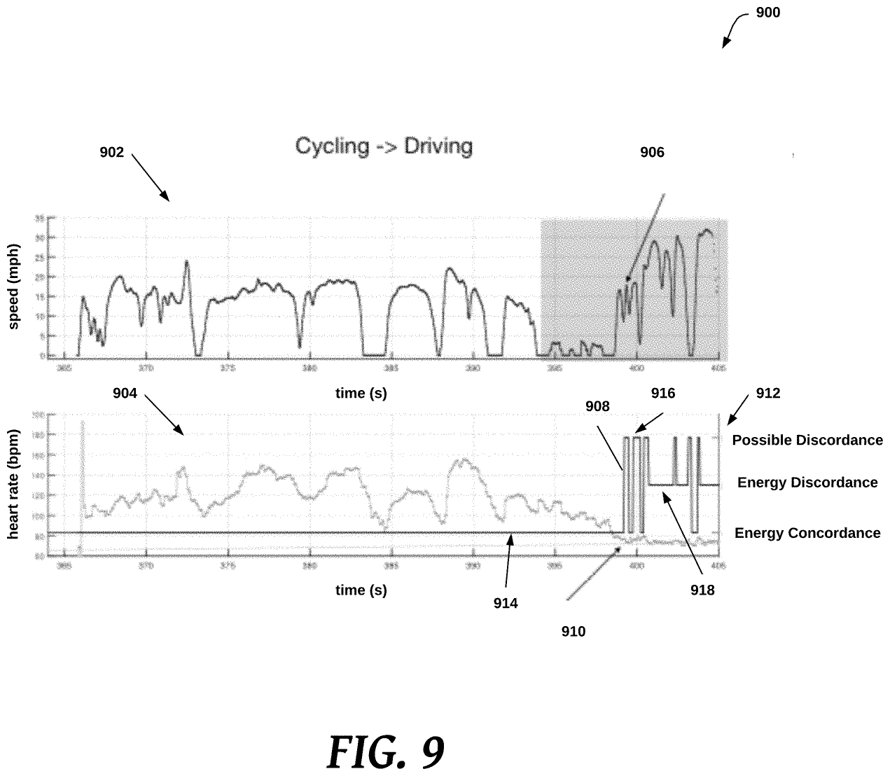

FIG. 9 shows sample heart rate and speed data that may be generated during a tracking session that includes a cycling activity followed immediately by a driving activity. The top panel 902 shows speed data 906 calculated by one or more speed models of the wearable device. The bottom panel 904 shows heart rate data 910 obtained from a heart rate sensor. The bottom panel 904 includes energy discordance segments 908 determined based on the speed data 906 and the heart rate data. The energy discordance segments 908 display the difference between the mechanical work rate calculated from the speed data 906 and the energy expenditure rate calculated from the heart rate data 908. The energy discordance segments 908 may be compared to the energy discordance threshold to detect an energy discordance.

As illustrated in the top panel 902, speed data 906 collected during the cycling activity may range from 0-25 miles per hour (mph), therefore slow driving may look like cycling based on the speed data 906. As shown in the bottom panel 904, the heart rate 910 of a user during the cycling activity may range from 90-150 beats per minute (bpm). The bottom panel 904 also includes the energy discordance segments 908 that display the difference between the mechanical work rate and the energy expenditure rate. To detect an energy discordance, the difference between the mechanical work rate and the energy expenditure rate reflected in the energy discordance segments 908 are compared to the energy discordance threshold 912. The energy discordance threshold 912 may range, for example, from 1-10. Energy discordance segments 908 falling below the energy discordance threshold 912 (e.g., near zero) indicate energy concordance (i.e., a small difference between the mechanical work rate and the energy expenditure rate). Energy discordance segments 908 falling within the energy discordance threshold 912 (e.g., between 1 and 10) indicate energy discordance (i.e., a significant difference between the mechanical work rate and the energy expenditure rate). Energy discordance segments 908 above the energy discordance threshold 912 (e.g., above 10) indicate possible energy discordance (i.e., a significant difference between mechanical work rate and energy expenditure rate that requires more data to confirm an if an energy discordance is observed.)

As shown on the right side of the bottom panel 904, energy concordance 914 (i.e., energy discordance segment 908 near 0) is observed during the cycling activity. The energy discordance segment 908 then rapidly fluctuates between energy concordance 914 (i.e., near 0) and possible discordance 916 (i.e., above 10) as the mechanical work rate and energy expenditure rate change rapidly during the transition period between cycling and driving. An energy discordance 918 (i.e., energy discordance segment within the discordance threshold) is then observed as the mechanical work rate and energy expenditure rate stabilize and the mechanical work rate exceeds the energy expenditure rate for a sustained period during driving.

Using energy discordance, the wearable device can detect the end of a cycling activity sooner than other techniques that rely only on speed and/or motion data. As shown in FIG. 9 the slow driving speed (e.g., below 20 mph) portion of the driving activity closely resembles the speed data during the cycling activity. By using energy discordance in addition to speed data and motion data to detect the end of cycling activities, the wearable device can detect the end of the cycling activity sooner and more accurately to exclude the slow driving portion form the cycling activity. Using energy discordance to precisely determine the end of a cycling activity prevents calculating performance information during non-cycling activities thereby increasing the accuracy of cycling performance information and accurate calibration of activity detection models, energy exertion models, cycling efficiency models, and other models included in the wearable device that may be tuned and/or calibrated using performance information.

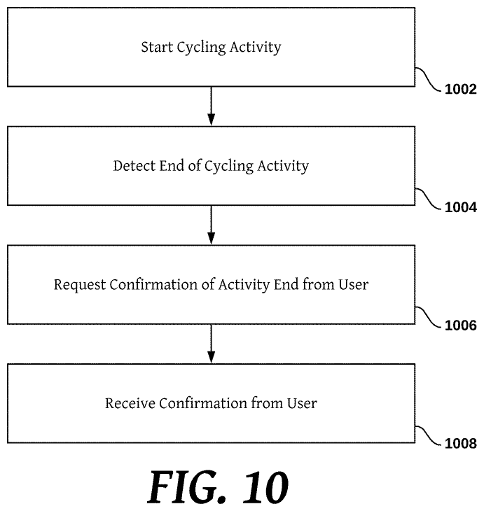

FIG. 10 illustrates an exemplary method of confirming the end of a cycling activity. At step 1002, a user starts a cycling activity. Once a cycling activity session is started, the wearable device records heart rate data form a heart rate sensor, motion data from one or more motion sensors, and pressure data from a pressure sensor. Data recorded by the arable device may be used to calculate user performance information including mechanical work rate, speed, and energy expenditure. At step 1004, the wearable device detects the end of the cycling activity based on one or more changes detected in user performance information (e.g. mechanical work rate below threshold, speed below cycling speed, energy expenditure below relative energy expenditure threshold, energy discordance between work rate and energy expenditure, and the like). Once the end of a cycling activity is detected, the wearable device may send a notification to a user including a confirmation request to confirm the user has ended the cycling activity at step 1006, The confirmation request may be displayed in a graphical user interface (GUI) rendered on a display screen of the wearable device. The confirmation request GUI may have a user input for receiving a user confirmation. At step 1008, the wearable device may receive confirmation from the user as an input into the confirmation request GUI.

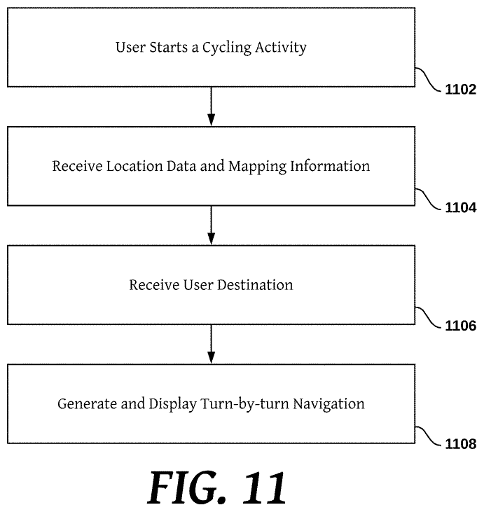

FIG. 11 illustrates an exemplary method of generating turn-by-turn navigation instructions for display on the wearable device during a cycling activity. At step 1102, a user starts a cycling activity. In various embodiments, the wearable device may receive location data from a GPS module and/or calculate location data from network connectivity information received from the communications interface, at step 1104. At step 1104, the wearable device may also receive mapping information from an external source (e.g., a mapping API, a remote computer server executing a mapping application, and the like). At step 1106, a user may enter a desired destination into a UI rendered by the wearable device. The desired destination may indicate a location where the user wants to travel by cycling. In response to receiving the desired destination, the wearable device may retrieve location information for the desired destination using mapping information obtained from the external source. The wearable device may also determine the relative distance between the user's current location and the desired destination by mapping the location data corresponding to the user's current location and the location of the desired destination using the mapping information.

The mapping information may also be used to generate the turn-by-turn navigation instructions to direct the user from the user's current location to the desired destination, at step 1108. In various embodiments, when multiple routes between the user's currently location and the desired destination are possible, the navigation instructions may optimize the generated navigation instructions based on traffic patterns, designated bike paths, bike friendly roads, and/or total elevation gained. The wearable device may output the turn by turn navigation instructions to a user in real time on a display as the user's current location information is updated. For example, after the user makes a turn included in the navigation instructions, the subsequent turn in the navigation instructions may be displayed on the user device. The wearable device may also update the turn-by-turn navigation instructions in real time based on the current location of the user. For example, if the user makes a wrong turn and/or starts cycling toward the desired destination on an alternate route, the wearable device may update the navigation instructions to direct the user from her updated current location.

The foregoing description is intended to convey a thorough understanding of the embodiments described by providing a number of specific exemplary embodiments and details involving activity detection, workout performance tracking, efficient use of battery and compute resources, power management in wearable devices, cycling activity monitoring, and turn-by-turn navigation for cycling. It should be appreciated, however, that the present disclosure is not limited to these specific embodiments and details, which are examples only. It is further understood that one possessing ordinary skill in the art, in light of known systems and methods, would appreciate the use of the invention for its intended purposes and benefits in any number of alternative embodiments, depending on specific design and other needs.

It is to he understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods, and systems for carrying out the several purposes of the disclosed subject matter. Therefore, the claims should be regarded as including such equivalent constructions insofar as they do not depart from the spirit and scope of the disclosed subject matter.

As used herein, the singular forms “a”, “an”, and “the” are intended to include the plural forms as well, unless the context clearly indicates otherwise. it will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

As used herein, the terms “and/or” and “at least one of” include any and all combinations of one or more of the associated listed items.

Certain details are set forth in the foregoing description and in FIGS. 1-11 to provide a thorough understanding of various embodiments of the present invention. Other details describing well-known structures and systems often associated with wearable devices, cycling activities, activity detection, workout performance tracking, efficient use of battery and compute resources, power management in wearable devices, cycling activity monitoring, turn-by-turn navigation for cycling, and the like, however, are not set forth below to avoid unnecessarily obscuring the description of the various embodiments of the present invention.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

1. A method for improving performance of a wearable device while recording a cycling activity, the method comprising:
starting a cycling activity;
receiving motion data of a user from a motion sensing module of the wearable device;
measuring, by a heart rate sensing module of the wearable device, a heart rate of the user, the heart rate sensing module comprising a photoplethysmogram (PPG) sensor configured to be worn adjacent to the user's skin;
calculating, by the one or more processor circuits, the user's performance information during the cycling activity, the performance information including a mechanical work rate and an energy expenditure rate; and
detecting, by the one or more processor circuits, an end of the cycling activity, the detecting the end of the cycling activity comprising:
comparing the mechanical work rate to a mechanical work rate threshold;
in response to detecting a value for the mechanical work rate above the mechanical work rate threshold, calculating a difference between the mechanical work rate and the energy expenditure rate; and
determine the end of the cycling activity based on the difference between the mechanical work rate the energy expenditure rate.

2. The method of claim 1, comprising:
receiving location data from a GPS module of the wearable device, the location data including a user speed of travel during the cycling activity;
receiving atmospheric pressure data from a pressure sensor of the wearable device; and
calculating the mechanical work rate based the location data, the atmospheric pressure data, and the motion data.

3. The method of claim 1, wherein the mechanical work rate threshold is consistent with light exercise.

4. The method of claim 2, comprising:
detecting, by the one or more processor circuits, a stop during the cycling activity, the detecting the stop comprising:
receiving a value for the user speed of travel below an expected cycling speed; and
receiving a motion data characterized as non-cycling motion.

5. The method of claim 4, comprising:
estimating an energy expenditure rate at two or more points during the cycling activity based on the user heart rate;
calculating a difference between a first energy expenditure rate estimated before the stop was detected and a second energy expenditure rate estimated after the stop was detected; and
detecting an intentional stop based on the difference between the first energy expenditure rate and the second energy expenditure rate.

6. The method of claim 5, comprising:
upon detecting the intentional stop, ending the cycling activity and terminating calculation of the user's performance information. The method of claim 5, comprising:
upon detecting the intentional stop, sending a confirmation request to a user to confirm the end of a cycling activity.

8. The method of claim 5, comprising:
detecting a temporary stop based on the difference between the first energy expenditure rate and the second energy expenditure rate; and
maintaining the cycling activity in response to detecting the temporary stop.

9. The method of claim 2, comprising:
receiving a value for the user speed of travel within an acceptable range for an expected cycling speed and motion data characterized as a cycling motion; and
identifying a driving activity based on the difference between the mechanical work rate the energy expenditure rate.

10. The method of claim 9, wherein the difference between the mechanical work rate and the energy expenditure rate is equivalent to the mechanical work rate minus an energy expenditure rate consistent with light exercise.

11. The method of claim 10, further comprising: in response to detecting the driving activity,
ending the cycling activity, and
stopping calculation of performance information.

12. The method of claim 2, wherein the user speed of travel is calculated, by the one or more processor circuits, from GPS positioning data received from the GPS module.

13. The method of claim 2, comprising:
receiving the atmospheric pressure data from the pressure sensor of the wearable device;
determining, by the one or more processor circuits, a grade describing a measure of steepness of terrain cycled on during the cycling activity; and
calculating, by the one or more processor circuits, elevation gained during the cycling activity using the grade.

14. The method of claim 13, comprising:
detecting, during the cycling activity, a stepping motion within the motion data, wherein the stepping motion is distinct from a pedaling motion;
extracting user steps included in the stepping motion using a user step model;
based on user steps and the grade, determining mechanical work performed during the stepping motion; and
incorporating mechanical work performed by the user during the stepping motion into the mechanical work rate.

15. The method of claim 14, wherein the performance information comprises at least one of an overall distance traveled, a total cycling time, a speed, the elevation gained, a power output, and a number of calories burned.

16. A method for improving performance of a wearable device while recording a cycling activity, the method comprising:
starting a cycling activity;
receiving motion data of a user from a motion sensing module of the wearable device;
measuring, by a heart rate sensing module of the wearable device, a heart rate of the user, the heart rate sensing module comprising a photoplethysmogram (PPG) sensor configured to be worn adjacent to the user's skin;
calculating, by the one or more processor circuits, the user's performance information during the cycling activity, the performance information including a mechanical work rate and an energy expenditure rate; and
detecting, by the one or more processor circuits, an end of the cycling activity, the detecting the end of the cycling activity comprising:
comparing the mechanical work rate to a mechanical work rate threshold; and
in response to detecting a value for the mechanical work rate below the mechanical work rate threshold, ending the cycling workout and stopping calculation of the user's performance information.

17. The method of claim 16, comprising:
generating, by the one or more processor circuits, a notification including a request to end the cycling workout, the notification displayed on a display of the wearable device.

18. The method of claim 16, wherein the motion data includes a step speed estimating a frequency of the user's lower body movements while performing pedal strokes during the cycling activity.

19. A system for improving performance of a wearable device while recording a cycling activity, the system comprising:
a motion sensing module configured to collect a user's motion data;
a heart rate sensing module configured to measure a heart rate of the user, wherein the heart rate sensing module comprises a photoplethysmogram (PPG) sensor and the PPG sensor is configured to be worn adjacent to the user's skin;
one or more processor circuits in communication with the motion sensing module and the heart rate sensing module, wherein the one or more processor circuits are configured to execute instructions causing the processor circuits to:
start a cycling activity;
calculate the user's performance information during the cycling activity, the performance information including a mechanical work rate and an energy expenditure rate;
compare the mechanical work rate to a mechanical work rate threshold;
in response to detecting a value for the mechanical work rate above the mechanical work rate threshold, calculate a difference between the mechanical work rate and the energy expenditure rate; and
detect an end of the cycling activity based on the difference between the mechanical work rate and the energy expenditure rate.

20. The system of claim 19, further comprising:
a GPS module configured to measure location data including a user speed of travel during the cycling activity;
a pressure sensor configured to measure atmospheric pressure data; and
wherein the processor circuits are further configured to:
receive the location data and the pressure data; and
calculate the mechanical work rate based the location data, the atmospheric pressure data, and the motion data.
